(19) United States
(12) Patent Application Publication
(10) Pub. No.: US 2014/0172099 A1
(43) Pub. Date: Jun. 19, 2014

(54) Techniques For Increasing The Acoustic Impedance Of The Cochlea's Round Window Membrane

(71) Applicant: OtoKinetics Inc., (US)

(72) Inventors: S. George Lesinski, Cincinnati, OH (US); Gregory N. Koskowich, Pleasanton, CA (US)

(73) Assignee: OTOKINETICS INC., Salt Lake City, UT (US)

(21) Appl. No.: 13/714,216

(22) Filed: Dec. 13, 2012

(51) Int. Cl. A61F 2/18; A61L 27/24

(52) U.S. Cl. CPC A61F 2/18; A61L 27/24
USPC 623/16.11; 623/23.72

(57) ABSTRACT

A method for increasing the acoustic impedance of the round window membrane of the cochlea in a human patient includes surgically opening access to the round window membrane and placing a packing layer over the round window membrane. Optionally a layer of collagen graft may be obtained from the patient and placed between the round window membrane and the packing layer. Optionally the packing layer may be enclosed within the collagen graft. Optionally bone paté may be obtained from the patient and used to form at least a portion of the packing layer. Optionally a silastic buttress may be placed under compression between the packing layer and the bone of sinus tympani of the patient.

### TECHNICAL FIELD

This disclosure relates to techniques for increasing the acoustic impedance of the cochlea's round window membrane.

### BACKGROUND

FIG. 1 is a layout drawing illustrating the construction of the human ear 10. Sound pressure energy enters the exterior ear 12 and through that the external auditory canal 14. The tympanic membrane (eardrum) 16 isolates the external auditory canal 14 from the middle and inner ear 18. The cochlea 20 which is part of the inner ear is physically coupled at its oval window 22 to the tympanic membrane 16 via the malleus bone 24, the incus bone 26 and the stapes bone 28. The tympanic cavity 30 is located in the middle ear within the temporal bone 31 on the inside side of the tympanic membrane 16. The round window 32 within the round window niche 32A in the cochlea 20 provides acoustic damping through deformation of the round window membrane 46 (which seals the round window 32) between the cochlea 20 and the tympanic cavity 30. The tympanic cavity 30 is also coupled to the Eustachian tube 34. The cochlea 20 is also coupled to the semicircular canals 36 (which provide the sense of motion) and the nerves of these are in turn coupled to the brain with the vestibular nerve 38. The nerves of the cochlea provide the sense of sound and are coupled to the brain with the cochlear nerve 40. The semicircular canals 36 and cochlea 20 are filled with lymphatic fluid.

FIG. 2 is a cut-away drawing illustrating the interaction of the round window, oval window and cochlea of the human ear 10. From this drawing is can be seen that the cochlea 20 is arranged with a single tube 42 that directs sound into the center of the cochlea 20 in a circular manner and then out again. Sound enters from the area of the oval window 14 and exits in the area of the round window 32 as illustrated by the arrows in FIG. 2.

FIG. 3 is a drawing illustrating surgical exposure of the round window 32 as it would be seen by partially removing (creating a temporary flap 44 in) the tympanic membrane 16.

### Overview

A method for increasing the acoustic impedance of the round window membrane of the cochlea in a human patient includes surgically opening access to the round window membrane and placing a packing layer over the round window membrane. Optionally a layer of collagen graft may be obtained from the patient and placed between the round window membrane and the packing layer. Optionally the packing layer may be enclosed within the collagen graft. Optionally bone paté may be obtained from the patient and used to form at least a portion of the packing layer. Optionally a silastic buttress may be placed under compression between the packing layer and the bone of sinus tympani of the patient.

### BRIEF DESCRIPTION OF THE DRAWINGS

The accompanying drawings, which are incorporated into and constitute a part of this specification, illustrate one or more examples of embodiments and, together with the description of example embodiments, serve to explain the principles and implementations of the embodiments.

In the drawings:

FIG. 1 is a layout drawing illustrating the layout of the human ear.

FIG. 2 is a cut-away drawing illustrating the interaction of the round window, oval window and cochlea.

FIG. 3 is a drawing illustrating surgical exposure of the round window.

FIG. 4 is a cross-sectional schematic drawing illustrating the layout of the round window niche.

FIG. 5 is a drawing illustrating a technique in accordance with one embodiment of the present invention for increasing the acoustic impedance of the round window.

FIG. 6 is a drawing illustrating a technique in accordance with another embodiment of the present invention for increasing the acoustic impedance of the round window.

FIG. 7 is a cross-sectional drawing illustrating a first surgical technique for increasing the acoustic impedance of the round window.

FIG. 8 is a cross-sectional drawing illustrating a second surgical technique for increasing the acoustic impedance of the round window.

FIG. 9 is a cross-sectional drawing illustrating a third surgical technique for increasing the acoustic impedance of the round window.

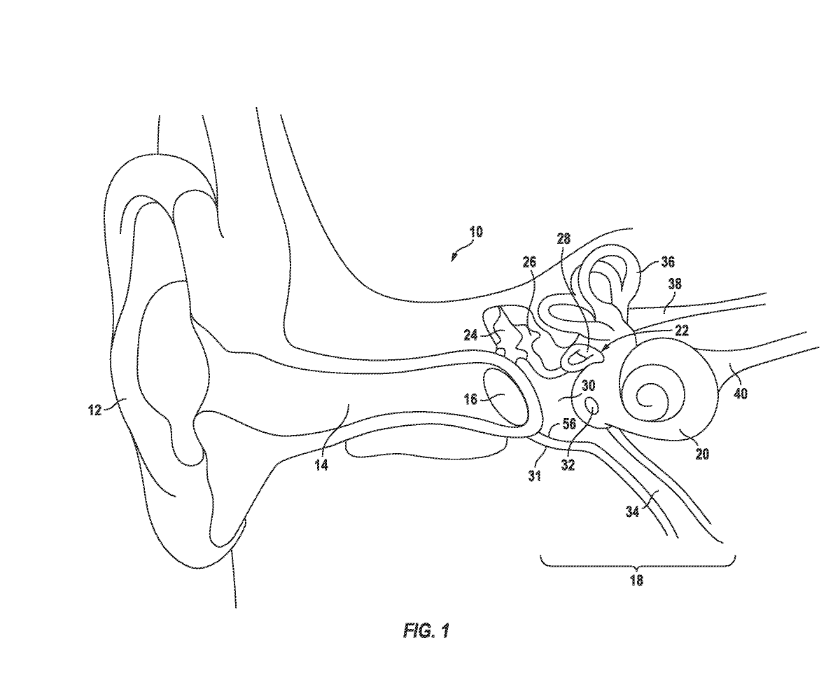

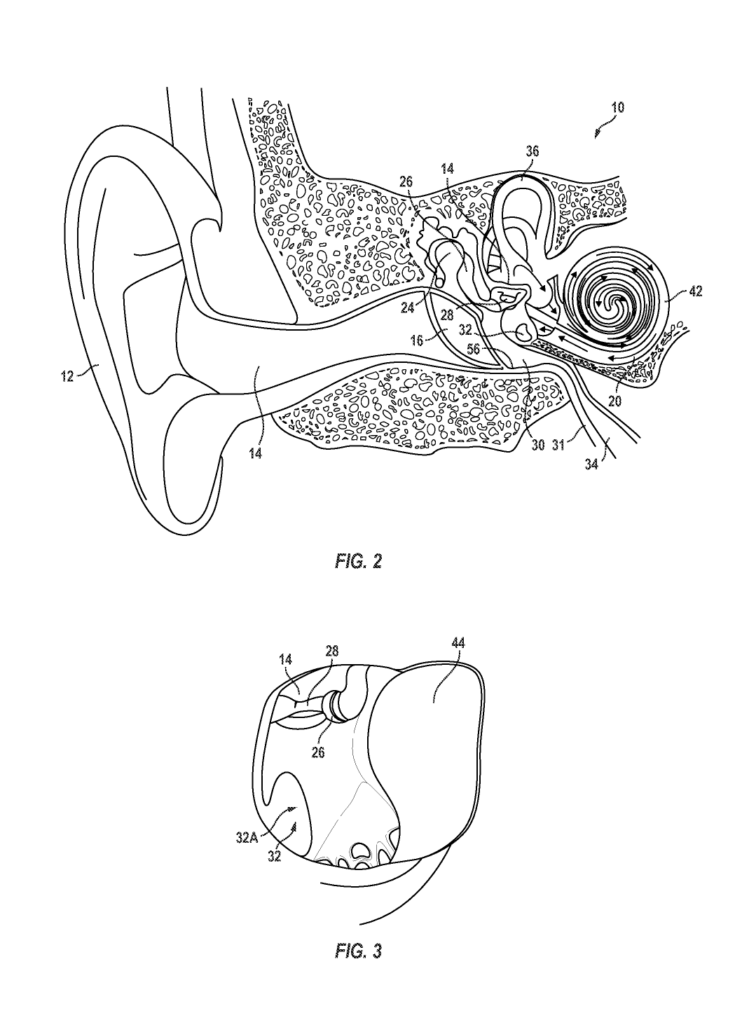

### DESCRIPTION OF EXAMPLE EMBODIMENTS

Example embodiments are described herein in the context of surgical techniques for increasing the impedance of the cochlea's round window membrane. Those of ordinary skill in the art will realize that the following description is illustrative only and is not intended to be in any way limiting. Other embodiments will readily suggest themselves to such skilled persons having the benefit of this disclosure. Reference will now be made in detail to implementations of the example embodiments as illustrated in the accompanying drawings. The same reference indicators will be used to the extent possible throughout the drawings and the following description to refer to the same or like items.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

References herein to “one embodiment” or “an embodiment” or “one implementation” or “an implementation” means that a particular feature, structure, part, function or characteristic described in connection with the embodiment can be included in at least one embodiment of the invention. The appearances of phrases such as “in one embodiment” or “in one implementation” in different places within this specification are not necessarily all referring to the same embodiment or implementation, nor are separate and alternative embodiments necessarily mutually exclusive of other embodiments.

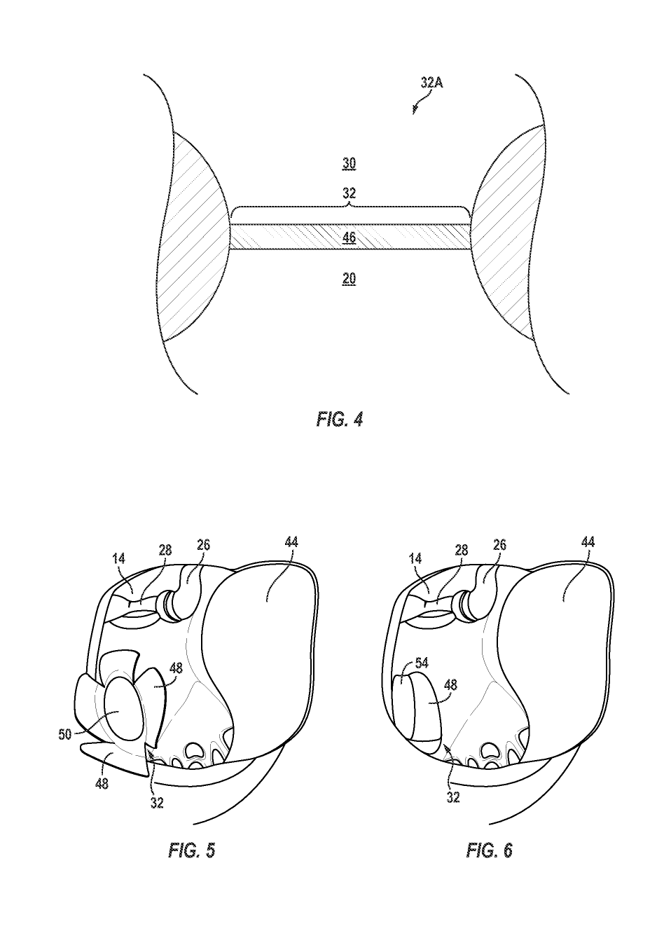

Turning to the figures, the round window membrane 46 (in FIGS. 4, 6, 7 and 8) is located within the round window niche 32A and is a flexible membrane that seals the round window 32 at the proximal end of the scala tympani portion of the cochlea 20. It permits the transmission of sound pressure waves (traveling waves) through the cochlear fluids (perilymph and endolymph) without compressing the fluid molecules. As otologic surgeons are developing new surgical procedures involving the inner ear (e.g., implanting sound amplifying transducers into the scala tympani portion of cochlea 20), there are instances when increasing the sound impedance of the round window membrane would be advantageous. By increasing the round window membrane acoustic impedance, less sound pressure energy would be disappated retrograde through deformation of the round window membrane and more sound pressure energy would be transmitted antegrade through the basal coil of the scala tympani to produce traveling waves of basilar membrane displacement that stimulate cochlear hair cells and the afferent auditory nerves. Thus the efficiency of an implanted transducer would be significantly increased. The flexible oval window 14 would now serve as the pressure release site.

As illustrated in FIG. 4, the round window membrane 46 is located in the round window niche 32A—a tiny “cave” on the posterior-inferior aspect of the promontory. It is readily identified in the middle ear through two different standard surgical approaches—a transcanal tympanotomy or a posterior tympanotomy through the facial recess. The round window membrane 46 lies underneath the superior boney lip of the round window niche 32A. The round window membrane 46 is oriented in a coronal plane, perpendicular to the promontory. Approximately 20% of individuals have a mucosal membrane in the niche lateral to the round window membrane that is easily removed with micro-otological surgical instruments to expose the round window membrane 46.

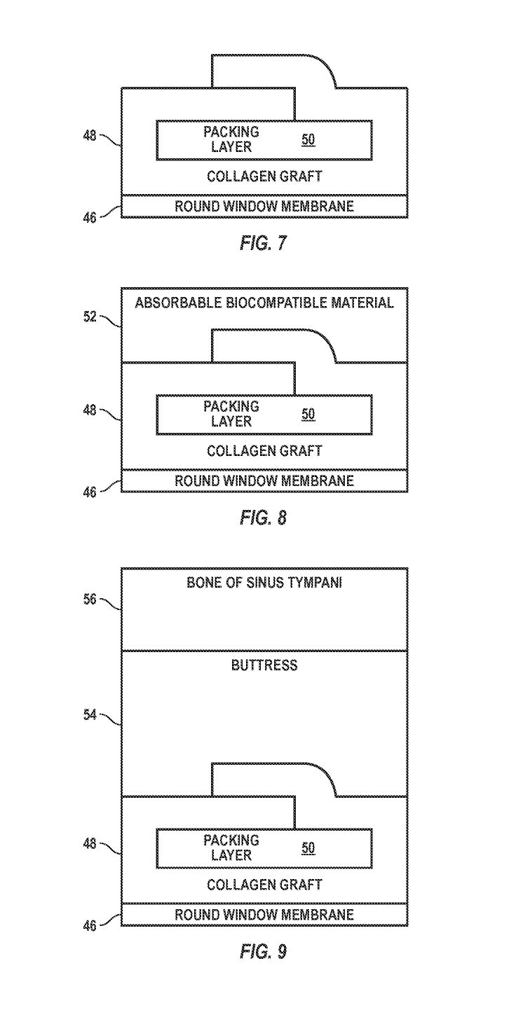

FIG. 5 is a drawing illustrating a technique in accordance with one embodiment of the present invention for increasing the acoustic impedance of the round window. FIG. 6 is a drawing illustrating a technique in accordance with another embodiment of the present invention for increasing the acoustic impedance of the round window. FIGS. 7, 8 and 9 are cross-sectional drawings illustrating first, second and third surgical techniques, respectively, for increasing the acoustic impedance of the round window. Several methods are now described for increasing the acoustic impedence of the round window membrane.

In accordance with a first embodiment illustrated in FIGS. 5 and 7, a thin collagen graft 48 obtained from the patient (isograft aerolar fascia, temporalis fascia, perichondrium or allograft collagen) is placed over the round window membrane 46 within the round window niche 32A. Graft 48 may be about the same thickness as round window membrane 46. Graft 48 will lay on round window membrane 46 and be disposed about the side of the round window niche 32A (the opening in the bone which the round window membrane 46 spans) and the corners will be visible to the surgeons when the packing layer 50 is in place (see FIG. 5). Collagen graft 48 may be a single piece that when folded on top envelops and encloses the packing layer as shown in FIG. 7. The collagen graft is not essential but helps to minimize the risk of tearing the round window membrane 46 when packing the niche. The round window niche is then packed with a packing layer 50 comprising a pledget of fat, loose collagen fibers, loose aurelear tissue and/or bone paté also obtained from the patient. For maximum round window acoustic impedence, optionally the round window on niche 32A may be entirely obliterated by forming the packing layer 50 with at least a portion of isograft bone paté 52. Bone paté is made, for example, by collecting bone chips in a sterile “trap” attached to the suction tubing while drilling the mastoid bone of the patient. The bone chips are saturated in a non-ototoxic antibiotic solution and then compressed into a paste. The bone paté is then packed into the round window niche 32A against the round window membrane 46 to form the packing layer 50 and fills the niche to obliterate the round window 32. The packing layer 50 is then enclosed with the remainder of the collagen graft layer 48 as illustrated in FIG. 7.

In accordance with another embodiment as illustrated in FIG. 8, the round window niche packing layer 50 may be tamponaded (secured in place) with one of several methods. These include placing a layer 52 of an absorbable biocompatable material over the packing layer 50. The absorbable biocompatible material may be one or more materials selected from hemostatic gauze, “Surgicel”, “Epidisc”, “Gelfoam”, and the like, which are used to temporarily tamponade the packing layer 50 until it is held in place by tissue healing.

In accordance with another embodiment as illustrated in FIG. 9, a more secure and permanent buttress layer 54 may be wedged between the packing layer 50 in the round window niche 32A and the bone of the sinus tympani 56. The buttress layer may comprise: a biocompatable silastic sponge (scleral sponge implanted for “scleral buckling”) which can be trimmed to fit, an Isograft or allograft cartilage strut, or a middle ear ossicular prosthesis (e.g., a hydroxylapatite, titanium or Teflon® TORP or PORP prosthesis), which would be reshaped to fit this narrow gap.

In accordance with the above-described embodiments, methods are provided to increase the round window membrane acoustic impedance through a range of stiffness from slight (e.g., fat or aerolar tissue) to complete fixation by entirely obliterating the round window niche with bone paté. The precise technique used by the surgeon will be dictated by the desired outcome for a particular patient.

While embodiments and applications have been shown and described, it would be apparent to those skilled in the art having the benefit of this disclosure that numerous modifications, variations and adaptations not specifically mentioned above may be made to the various embodiments of the invention described herein without departing from the scope of the invention which is defined in the claims.

What is claimed is:

1. A method for increasing the acoustic impedance of the round window membrane of the cochlea in a human patient, the method comprising:
surgically opening access to the round window membrane;
placing a packing layer over the round window membrane.

2. The method of claim 1, further comprising:
obtaining a collagen graft from the patient;
placing the collagen graft between the round window membrane and the packing layer.

3. The method of claim 1, further comprising:
obtaining bone material from the patient;
forming a bone paté out of the bone material;
forming the packing layer to comprise bone paté.

4. The method of claim 2, further comprising:
obtaining bone material from the patient;
forming a bone paté out of the bone material;
forming the packing layer to comprise bone paté.

5. The method of claim 1, further comprising:
placing a layer of absorbable biocompatible material over the packing layer.

6. The method of claim 2, further comprising:
placing a layer of absorbable biocompatible material over the packing layer.

7. The method of claim 3, further comprising:
enclosing the packing layer within the collagen graft.

8. The method of claim 4, further comprising:
enclosing the packing layer within the collagen graft.

9. The method of claim 5, further comprising:
enclosing the packing layer within the collagen graft.

10. The method of claim 6, further comprising:
enclosing the packing layer within the collagen graft.

11. The method of claim 1, further comprising:
placing a silastic buttress layer under compression between the packing layer and the bone of sinus tympani of the patient.

12. The method of claim 2, further comprising:
placing a silastic buttress layer under compression between the packing layer and the bone of sinus tympani of the patient.

13. The method of claim 11, further comprising:
enclosing the packing layer within the collagen graft.

14. The method of claim 12, further comprising:
enclosing the packing layer within the collagen graft.
